(19) United States
(12) Patent Application Publication
(10) Pub. No.: US 2025/0288558 A1
(43) Pub. Date: Sep. 18, 2025

(54) THERAPEUTIC FOR SPINAL CORD INJURY TO INDUCE HYPOTHERMIA

(71) Applicant: The Trustees of Indiana University, Bloomington, IN (US)

(72) Inventors: Lingxiao Deng, Carmel, IN (US); Xiang Gao, Whitestown, IN (US)

(21) Appl. No.: 19/077,737

(22) Filed: Mar. 12, 2025

Related U.S. Application Data
(60) Provisional application No. 63/564,919, filed on Mar. 13, 2024.
(60) Provisional application No. 63/683,933, filed on Aug. 16, 2024.

(51) Int. Cl. A61K 31/4174; A61P 25/00

(52) U.S. Cl. CPC A61K 31/4174; A61P 25/00

(57) ABSTRACT

Disclosed herein are methods of treating a traumatic injury by pharmacologically inducing hypothermia. The traumatic injury can involve any of spinal cord injury, traumatic brain injury (TBI), and stroke. Also disclosed are methods of mitigating secondary injury following traumatic injury due to deleterious cellular processes, such as injury-activated cell signaling, by drug-induced hypothermia. Disclosed methods involve administering a pharmacological agent, such as an adrenergic agonist, to a subject post-trauma, thereby improving functional recovery outcomes.

### CROSS-REFERENCE TO RELATED APPLICATIONS

This application claims the benefit under 35 U.S.C. § 199(e) of U.S. Provisional Application No. 63/564,919 entitled THERAPEUTIC FOR SPINAL CORD INJURY TO INDUCE HYPOTHERMIA, filed on Mar. 13, 2024 and of U.S. Provisional Application No. 63/683,933 entitled THERAPEUTIC FOR SPINAL CORD INJURY TO INDUCE HYPOTHERMIA, filed on Aug. 16, 2024, both of which are incorporated by reference in their entirety.

### STATEMENT OF GOVERNMENTAL RIGHTS

This invention was made with government support HT9425-23-1-0700 awarded by the Medical Research and Development Command. The Government has certain rights in the invention.

### FIELD OF THE INVENTION

The present disclosure relates generally to methods of treating a traumatic injury, for example, trauma involving spinal cord injury, by pharmacologically inducing hypothermia. Disclosed treatment strategies are suitable for immediate application post-injury. The present disclosure also relates to methods of mitigating detrimental cellular processes triggered post-trauma, including neurological damage.

### BACKGROUND

Spinal cord injury (SCI) is a devastating condition of motor, sensory, and autonomic dysfunction. The significant costs associated with the management and lifetime care of SCI patients also presents a major economic burden in the United States alone, there are currently over 300,000 Americans suffering from a SCI with about 18,000 new cases annually. To date, no effective treatment is available to achieve the significant functional recovery after SCI.

Given the challenges in achieving regeneration of the injured spinal cord, neuroprotection has been at the forefront of clinical translation. Systematic application of hypothermia in the acute phase post-SCI has been shown to be neuroprotective, i.e., limit secondary injury, in both experimental animal and human studies. However, practical application of conventional methods to induce hypothermia are limited by several unsolved problems, including a narrow therapeutic window for optimal efficacy, temporal or limited protection, and severe side effects.

There is a need to develop and translate neuroprotective strategies that can improve outcomes following SCI, such as hypothermic induction. For example, strategies that extend the therapeutic window, allow for immediate, on-site care are needed. Additionally, neuroprotective strategies that can be combined for potential synergistic effect may also contribute to improved therapeutic strategies. Accordingly, there exists a need to provide effective treatment strategies with immediate applicability post-injury. Additionally, there exists a need for novel therapeutic interventions to delay the physiological progression of secondary injury to mitigate SCI-induced neurological dysfunction-following SCI. Aspects of the invention disclosed herein address these needs.

### SUMMARY OF THE INVENTION

A first aspect of the invention includes methods for inducing hypothermia in a subject who has suffered a traumatic injury, such as to mitigate, prevent, or interfere with damaging, post-traumatic cellular processes.

A first embodiment is a method for inducing hypothermia in a subject post-trauma, including administering an effective amount of an adrenergic agonist to the subject, thereby inducing hypothermia in the subject, where the adrenergic agonist is administered to the subject after the subject has suffered an injury.

A second embodiment is a method where inducing hypothermia includes reducing the subject's core body temperature to a hypothermic temperature of about 30° C.-35° C., 30° C.-34° C., 30° C.-33° C., 30° C.-32° C., 31° C.-35° C., 31° C.-34° C., 31° C.-33° C., 32° C.-35° C., 32° C.-34° C., or 32° C.-33° C., where each range is inclusive.

A third embodiment is a method where the hypothermic temperature is maintained for 0.5 to 6 hours, 0.5 to 12 hours, 0.5 to 24 hours, 6 to 12 hours, 6 to 24 hours, 6 to 8 hours, 2 to 6 hours, or 3 to 5 hours, where each range is inclusive, after administration of an adrenergic agonist.

A fourth embodiment is a method where after maintaining the hypothermic temperature, the method further includes applying heat to the subject until the subject's core body temperature reaches 36.5-38.5° C., inclusive, or allowing the subject's core body temperature to reach 36.5-38.5° C., inclusive, without intervention.

A fifth embodiment is a method including inducing hypothermia in a subject while the subject is in an ambient temperature environment in the range of 18° C. to 40° C., 18° C. to 37° C., 18° C. to 35° C., 25° C. to 35° C., 22° C. to 30° C., 22° C. to 27° C., 23° C. to 27° C., 24° C. to 27° C., or 22° C. to 25° C., where each range is inclusive.

A sixth embodiment is a method where the injury includes traumatic brain injury, a stroke, a spinal cord injury, or a combination thereof.

A seventh embodiment is a method including administering the adrenergic agonist to the subject about 1 to 24 hours, 1 to 16 hours, 1 to 12 hours, 0.5 to 1 hour, 0.5 to 2 hours, 0.5 to 3 hours, 1 to 8 hours, 2 to 6 hours, 3 to 5 hours, or 0 to 60 minutes after the injury, where each range is inclusive.

An eighth embodiment is a method where the adrenergic agonist includes p-aminoclonidine, lidamidine, tizanidine, moxonidine, xylazine, detomidine, medetomidine, dexmedetomidine, a pharmaceutical salt thereof, an analog thereof, or a combination thereof.

A ninth embodiment is a method where the adrenergic agonist includes an alpha2-adrenergic receptor agonist.

A tenth embodiment is a method where the alpha2-adrenergic receptor agonist includes dexmedetomidine hydrochloride.

An eleventh embodiment is a method including administering the adrenergic agonist to the subject by parenteral administration, oral administration, mucosal administration, subcutaneous administration, intravenous administration, intramuscular administration, intranasal administration, or a combination thereof.

A twelfth embodiment is a method including administering the adrenergic agonist to the subject by intravenous infusion.

A thirteenth embodiment is a method including administering the adrenergic agonist to the subject in a single dose or more than one dose.

A fourteenth embodiment is a method where the subject exhibits improved motor function, cognitive function, sensory function, autonomic function, sexual function, or a combination thereof, after induction of hypothermia, relative to motor function and/or cognitive function after the injury and prior to induction of hypothermia.

A fifteenth embodiment is a method where the subject is a mammal.

A sixteenth embodiment is a method where the mammal is a dog, a cat, a horse, or a human.

A seventeenth embodiment is a method of treating a post-trauma injury in a subject, where at least one of a single dose at 8 mcg/kg and multiple doses at 0.2 to 2.5 mcg/kg per hour of an adrenergic agonist is administered to the subject after the subject has suffered an injury.

An eighteenth embodiment is a method of inducing hypothermia where the subject's core body temperature is reduced to a hypothermic temperature of about 30° C.-35° C., 30° C.-34° C., 30° C.-33° C., 30° C.-32° C., 31° C.-35° C., 31° C.-34° C., 31° C.-33° C., 32° C.-35° C., 32° C.-34° C., or 32° C.-33° C.

A nineteenth embodiment is a method of inducing hypothermia in a subject following a post-trauma injury where the a alpha2-adrenergic receptor agonist, such as dexmedetomidine hydrochloride is administered to the subject.

### BRIEF DESCRIPTION OF THE DRAWINGS

The above-mentioned and other features and advantages of this disclosure, and the manner of attaining them, will become more apparent and will be better understood by reference to the following description of exemplary embodiments taken in conjunction with the accompanying drawings, wherein:

FIG. 1 is a schematic diagram illustrating the methods and potential mechanisms of action of the methods described herein.

FIG. 2A is a graph showing the change in temperature over time after one dose of Dex (100 μg/kg) IP injection in c57 BL/6 mice at ambient temperature of 24.5° C. Dex induced moderate to modest hypothemia. The duration of hypothemia increased from 2 hr to 6 hr when combined with 2.5% of Avertin, while Avertin itself just reduced the body temperature slightly.

FIG. 2B is a graph showing body temperature over time when Dex (100 μg/kg) was administered 1 hour after T9 SCI. It induced up to 16 hr moderate hypothermia. Then the injured mice slowly and spontaneously rewarmed to normal body temperature at 24 hr post trauma with the rate of <1.0° C. per hour.

FIG. 3A is a schematic drawing of behavior test timeline.

FIG. 3B is a graph depicting the results of the Basso Mouse Scale (BMS) test. The Dex treated group had better locomotion recovery compared to none-treatment SCI group with significant higher score. (female, n=10, *p<0.05, **p<0.01)

FIG. 3C is a graph depicting the results of the Rotarod test. The results showed a significant increase of coordination and balance in Dex treated SCI mice compared to none-treated SCI mice. (female, n=10, *p<0.05, **p<0.01)

FIG. 3D is a graph depicting the results of the Grid Walk test. The results showed the better recovery of sensorimotor functions in Dex treated group. (female, n=10, *p<0.05, **p<0.01)

FIG. 4 is graph of a metabolic cage experiment urine volume results for a sham mouse, a mouse with a SCI, and a mouse with a SCI and treated with Dex.

FIG. 5 is a void spot assay of urine output of a sham mouse, a mouse with a SCI, and a mouse with a SCI and treated with Dex.

FIG. 6A is graph of volume of urine urinated by a sham mouse, a mouse with a SCI, and a mouse with a SCI and treated with Dex 4 hours after a 1 mL saline IP injection.

FIG. 6B is a graph of the number of times a sham mouse, a mouse with a SCI, and a mouse with a SCI and treated with Dex urinates 4 hours after a 1 mL saline IP injection.

FIG. 7A are bar graphs depicting measurement results presented in total area of spinal cord coronal section among sham, SCI and SCI+Dex groups. Compared to sham group SCI injury had a drastic impact on both SCI and SCI+Dex groups with significant decrease in the total area in these two groups that was not only in epicenter, but also in remote region (2.5 mm away from impact site.) (female, n=3, *p<0.05, ***p<0.005).

FIG. 7B are bar graphs of quantification data demonstrating that although there was no significant difference regarding the total area of spinal cord section between SCI and SCI+Dex groups, significant more white matter was preserved with Dex treatment, especially in epicenter and its surrounding area. (female, n=3, *p<0.05, ***p<0.005).

FIG. 8A are graphs depicting neuron cell counting results. (female, n=3, *p<0.05, **p<0.01, ***p<0.005).

FIG. 8B are graphs quantifying the axons preserved. (female, n=3, *p<0.05, **p<0.01, ***p<0.005).

FIG. 9 are bar graphs illustrating GFAP intensity measurement results. (female, n=3, *p<0.05, **p<0.01, ***p<0.005).

FIG. 10A is a graph depicting the 24 hour body temperature curve of different treatment groups post-surgery. (female, n=3, *SCI vs. SCI+Dex, *p<0.05, ***p<0.005, #SCI+CH vs. SCI+Dex, #p<0.05, ###p<0.005).

FIG. 10B is a graph depicting the BMS test scores for each of the treatment groups. (female, n=3, *SCI vs. SCI+Dex, *p<0.05, ***p<0.005, #SCI+CH vs. SCI+Dex, #p<0.05, ###p<0.005).

FIG. 10C is a graph depicting the Rotatard test scores for each of the treatment groups. (female, n=3, *SCI vs. SCI+Dex, *p<0.05, ***p<0.005, #SCI+CH vs. SCI+Dex, #p<0.05, ###p<0.005).

FIG. 10D is a graph depicting the Grid Walk scores for each of the treatment groups. (female, n=3, *SCI vs. SCI+Dex, *p<0.05, ***p<0.005, #SCI+CH vs. SCI+Dex, #p<0.05, ###p<0.005).

FIG. 11 is a schematic chart of the multiple functional tests and analysis performed on the treatment groups.

### DETAILED DESCRIPTION

The present disclosure is directed to a use and a method of administering an effective amount of an adrenergic agonist to a subject to induce hypothermia in the subject, wherein the subject has suffered an injury. The injury may be a traumatic brain injury, a stroke, a spinal cord injury, or a combination thereof.

A spinal cord injury (SCI) comprises damage to the spinal cord resulting in a loss of function, such as mobility or feeling. SCI is initially caused by external mechanical impact, which results in cell deformation and death. The injury can be complete, where there is a total loss of sensory and motor function below the injury site, or incomplete, where there is some remaining function. The extent of the injury and the specific functions affected depend on the location and severity of the damage.

While SCI is initially caused by external mechanical impact resulting in cell deformation and death, the damage is further propagated by secondary injury mechanisms including ischemia, sodium and calcium-mediated cell injury, glutamatergic excitotoxicity, hemorrhage, and inflammation. The secondary injuries may amplify the primary damage and promote cystic degeneration and glial scar formation, thereby preventing functional recovery.

Systemic hypothermia is a medical condition in which the subject's core body temperature drops below 35° C. Systemic hypothermia is a proven neuroprotective strategy to limit secondary injury in animal models of acute spinal cord injury. By inducing hypothermia, one or more of the following mechanisms of secondary injury are decreased and/or inhibited: the rate of metabolism is reduced, the generation of free radicals is decreased, excitotoxicity and neuron apoptosis is inhibited, the Blood Spinal Cord Barrier is preserved, edema is prevented, astrogliosis is inhibited, angiogenesis is increased, axonal damage is decreased, and neurogenesis is promoted.

Traditional methods of intervention to delay the progression of the pathophysiological processes associated with secondary injury include inducing hyperthermia in a clinical setting via surface cooling or endovascular cooling. Surface cooling is most commonly performed applying a cooling blanket or ice to the patient. While simple to perform, surface cooling takes an extended amount of time to drop the patient's internal core temperature sufficiently to induce hyperthermia. This delay in inducing hyperthermia is in addition to the amount of time needed to get an injured subject to a clinical setting. Additionally, external cooling does not always result in a sufficient reduction of the core temperature of the patient. Endovascular cooling is a rapid-cooling technique in which a balloon catheter is inserted in a central vein of the patient with a “closed loop” internal cooling circuit, the catheter then cools the patient's blood as it circulates past the catheter. Endovascular cooling carries risks of deep venous thrombosis and catheter site infection as well as the difficulty to perform such cooling in the field (at the location the subject was injured).

An adrenergic agonists is a drug that stimulates a response from the adrenergic receptors. Adrenergic agonists may be used to induce hypothermia. Adrenergic agonists may include p-aminoclonidine, lidamidine, tizanidine, moxonidine, xylazine, detomidine, medetomidine, dexmedetomidine, a pharmaceutical salt thereof, an analog thereof, and combinations thereof. Preferably, the adrenergic agonist may be a alpha2-adrenergic receptor agonist, such as dexmedetomidine hydrochloride (Dex). The FDA-approved sedative drug Dex can be used to modulate body temperature, in the absence of auxiliary heating and cooling equipment, indicating that Dex can be used for on-site, immediate induction of hypothermia.

Dex is indicated for sedation and does not cause respiratory depression. It is commonly used during sedation of intubated and mechanically ventilated subjects and peri-procedural sedation of non-intubated subjects. Dex inhibits central sympathetic outflow by blocking the alpha receptors in the brainstem, inhibiting the release of norepinephrine. Due to the mechanism of action, Dex has analgesic properties that have lead to off-label use as a treatment and prevention of delirium, adjunctive analgesia, therapy for insomnia, and treatment of alcohol withdrawal.

Further, it has been shown that administration of Dex induces modest hypothermia (30-32° C.) in one hour at ambient temperatures (24-25° C.). This modulation of body temperature does not require any auxiliary heating and cooling equipment, allowing Dex to be used to induce hypothermia on-site at the location of injury.

Dex may be administered to mammals, such as a dog, cat, horse, and a human. Administration may be by parenteral administration, oral administration, mucosal administration, subcutaneous administration, intravenous administration, intramuscular administration, intranasal administration, buccal administration sublingual film administration or a combination thereof. Preferably, the administration is intravenous or intramuscular. To induce hypothermia, a single dose of the drug may be administered. In an alternative embodiment, multiple doses of the drug, such as two doses, three doses, four doses, or any other effective number of doses may be administered. The multiple doses of the drug may comprise an initial bolus of the drug, followed by maintenance doses over a period of time. Alternatively, administration of a loading dose followed by a continuous infusion titrated to the desired level of hypothermia may also be used.

To induce hypothermia, Dex may be administered via intraperitoneal or intramuscular injection with a single dose of about 8 mcg/kg. If multiple doses are administered to induce hypothermia, Dex may be administered at a dosage range of about 0.2 to about 2.5 mcg/kg per hour per dose. Preferably the dosage range is about 0.2 to about 1.5 mcg/kg per hour. Most preferably the dosage range is about 0.2 to about 0.7 meg/kg per hour. If a loading dose is used, Dex may be administered as an initial dose of about 0.5 to about 1.0 mcg/kg followed by a continuous infusion of about 0.2 to about 0.7 mcg/kg per hour titrated to the desired hypothermia goals. Preferably, the total dose of Dex is about 400 mcg for a 50 kg patient to induce hypothermia.

To induce hypothermia, Dex may be administered within a time from injury from 0 hours, 0.5 hours, 1 hour, 2 hours, or 3 hours to 4 hours, 5 hours, 6 hours, 12 hours, or 24 hours, or any range using any of the foregoing values as endpoints, such as 0 hours to 24 hours, 0.5 hours to 12 hours, 1 hour to 6 hours, 2 hours to 5 hours, or 3 hours to 4 hours, wherein each range is inclusive.

The subject's core temperature may be reduced with administration of the adrenergic agonist while the subject is at ambient temperatures. Ambient temperatures may range from 18° C., 22° C., or 25° C. to 30° C., 35° C., or 40° C., or any range using any of the foregoing values as endpoints, such as 18° C. to 40° C., 22° C. to 35° C., or 25° C. to 30° C., wherein each range is inclusive.

To get the neuroprotective effects of the adrenergic agonist, a subject's core body temperature may be reduced to about 30° C., 31° C., or 32° C. to 33° C., 34° C., or 35° C., or any range using any of the foregoing values as endpoints, such as 30° C. to 35° C., 31° C. to 34° C., or 32° C. to 33° C., wherein each range is inclusive.

This hypothermic temperature may be maintained in the subject for an amount of time from 0.5 hours, 2 hours, 3 hours, or 6 hours to 8 hours, 10 hours, 12 hours, or 16 hours, or any range using any of the foregoing values as endpoints, such as 0.5 hours to 16 hours, 2 hours to 12 hours, 3 hours to 10 hours, or 6 hours to 8 hours, wherein each range is inclusive.

Once the hypothermic treatment time has elapsed, the subject may be rewarmed. A subject may be rewarmed by applying heat to the subject or allowing the subject to warm without intervention until the subject's core body temperature reaches a temperature from 36° C., 36.5° C., or 37° C. to 37.5° C., 38° C., or 38.5° C., or any range using any of the foregoing values as endpoints, such as 36° C. to 38.5° C., 36.5° C. to 38° C., or 37° C. to 37.5° C., wherein each range is inclusive.

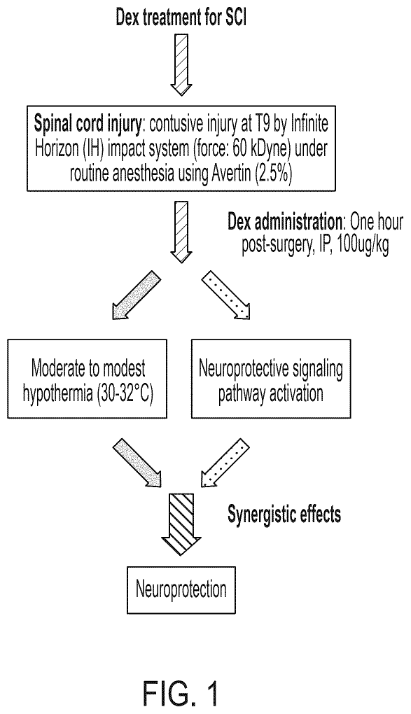

While not being bound by any single scientific theory, as shown in FIG. 1, neuroprotection may be achieved by Dex in a subject suffering from SCI through the synergistic effect of inducing moderate hypothermia (30-32° C.) and activation of the ERK signaling pathway. The neuroprotective properties of Dex via activating ERK signaling pathway, has been reported in several other injury models. Dex treatment may elevate the phosphorylated level of ERK1/2 in vitro. Therefore, in a SCI, Dex treatment may show a strong neuroprotective effect might via a synergistic mechanism of its dual roles in hypothermia induction and inherent ERK signaling pathway activation.

As shown in the example below, by using administration of an adrenergic agonist, such as Dex, a subject may exhibit improved motor function, cognitive function, sensory function, autonomic function, sexual function, or a combination thereof, after induction of hypothermia, relative to motor function and/or cognitive function after the injury and prior to induction of hypothermia. Dex treatment after an injury may also improve lower urinary tract dysfunction, reduce lesion area, attenuate glial scar formation, and increase in spared spinal cord tissue, spinal cord neurons, and axons. Dex may also activate multiple signaling pathways, which are neuroprotective. A synergistic interaction between induced hypothermia and activated neuroprotective signaling pathways may contribute to the beneficial effects of treating a subject with Dex after SCI.

Hypothermic induction with administration of an adrenergic agonist may drastically increase neuroprotection with significantly enhanced locomotion recovery (up to 75% increasing compared to non-treatment, 3 times than average improvement of other reports). The improvement may persist at least 8 weeks after the SCI. Further, diminishing Dex induced hypothermia (such as by applying heating-pad) may only partially hinder the neuroprotective effects of Dex, strongly suggesting the existence of “other” neuroprotective property of Dex.

### EXAMPLES

### Example 1: Inducing Hypothermia in Mouse Model

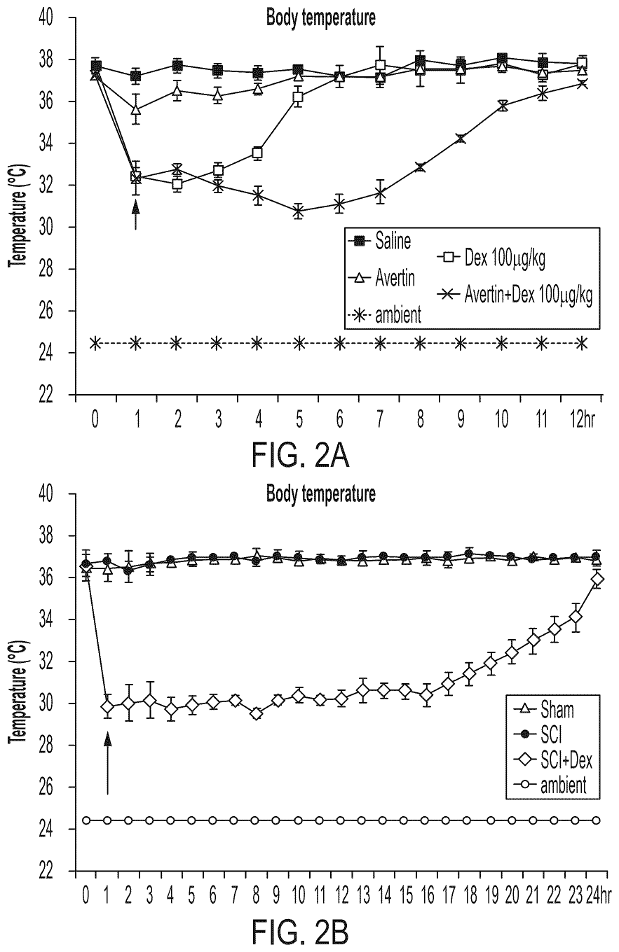

The effects of different doses of Dex on hypothermia induction in none-injury young adult c57 BL/6 mice was tested. By intraperitoneal (IP) injection, lower dose of Dex (1, and 10 μg/kg) failed to induce hypothermia. When the dose of Dex raised up to 50 μg/kg, a very short period (less than one hour) of mild (34-35° C.) hypothermia could be rapidly induced at ambient temperature of 24.5° C. (FIG. 2A). When the dose increased to 100 μg/kg, a 3 hour durable mild to moderate (30-32° C.) hypothermia had been successfully induced (FIG. 2A). However, when the dose of Dex reached 200 μg/kg, more severe hypothermia (lower than 28° C.) had been induced and it caused death in about 40% of the treated mice due to urethral obstructions. Further, we examined the change of Dex induced hypothermia when the routine Avertin (2.5%), a wildly used anesthetic for small animal surgery was applied. Avertin itself did not significantly induce hypothermia. The body temperature of Avertin-only mice decreased slightly and return to the normothermia quickly at ambient temperature of 24.5° C. (FIG. 2A). If different dose of Dex was applied one hour post Avertin administration, induced hypothermia increased from less than one hour to 2 hours in none-injury mice with Dex (50 μg/kg) treatment and extended from 3 hours to 6 hours with Dex (100 μg/kg) treatment, respectively. The average hypothermia time for SCI study was about 4 hours, and 6 hour mild to moderate hypothermia induced by combining Avertin and Dex was long enough to cover this period. Thus, the 100 μg/kg dose of Dex was chosen for further study. More interestingly, the administration of Dex (100 μg/kg) one hour after moderate contusive spinal cord injury (SCI) at thoracic 9 (T9) using LISA impact, extended the duration of hypothermia up to 16 hours. The injured animals then slowly and spontaneously rewarmed back to the normal temperature (˜37.50 C) in 5-6 hours (FIG. 2B, <1.00 C per hour). No auxiliary heating and cooling equipment was needed to induce and maintain the hypothermia during whole process. These results suggest that Dex could be potentially applied on-site to induce early hypothermia easily after SCI.

### Example 2: SCI Treatment with Dex in a Mouse

In a mouse model of moderate contusive SCI under routine anesthetic (Avertin), one dose (100 mcg/kg) of Dex was administered via intraperitoneal injection. To translate human doses to mice, a typical conversion factor based on body surface area was used, which is approximately 12.3 times the human dose for mice. Thus, the 100 mg/kg dose in mice equates to roughly 8 mcg/kg for a human. The 100 mcg/kg single dose in the mouse study, aligns well with the total dose used in human patients, which is around 400 mcg for a 50 kg individual.

Hypothermia in the mouse was induced rapidly. Lower urinary tract dysfunction is a common outcome of SCI. Therefore, urinary tract dysfunction in the SCI animal model was used for evaluation of any effect of Dex following SCI. The effectiveness of Dex induced hypothermia may allow for recovery weeks after SCI injury. For example, improvements to a mouse with an SCI treated with Dex may be seen at least 6 weeks post-SCI injury (such as improvements in cystometric measurements, metabolic cage results, rotarod results, urine spot assays, etc.).

### Example 3: Impact of Dexamedetomidine on Functional Outcomes After SCI

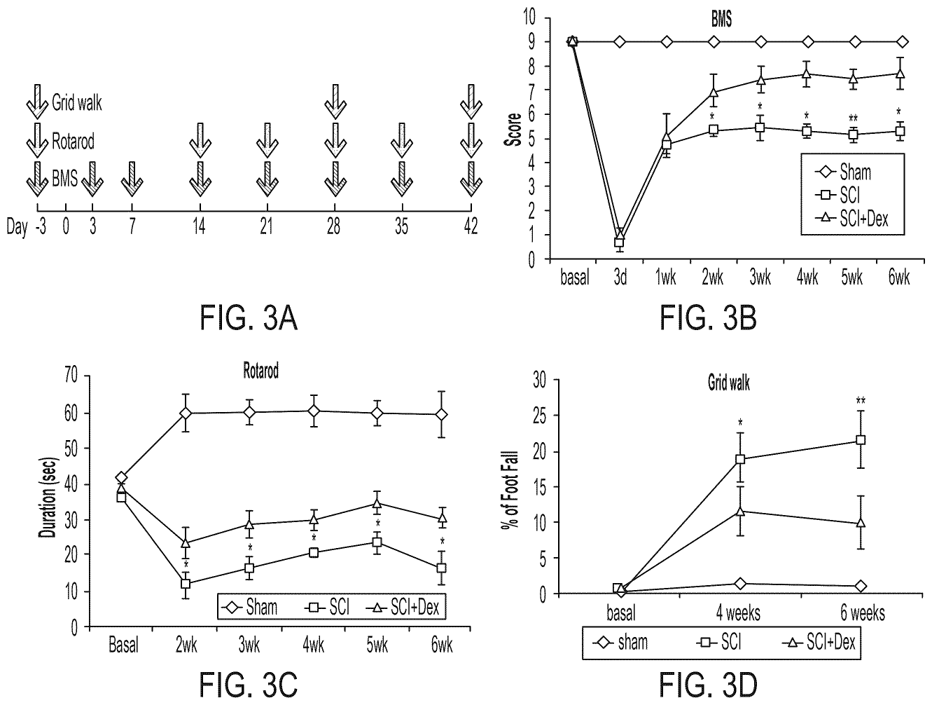

Dex may be neuroprotective by effectively inducing therapeutic hypothermia in a SCI. Therefore, we compared the functional outcomes of Dex treated SCI mice with SCI only mice using multiple behavior tests post-trauma. C57 BL/6 wild type mice (female) at age of 8 weeks were randomly assigned into three different groups, including sham, SCI, and SCI+Dex. Then the moderate T9 contusive SCI was introduced in two injury groups using LISA injury model. Mice in sham and SCI only group were put on the heating pad to maintain their normal body temperature, while mice in Dex treated group were exposed in ambient room temperature of 24.5° C. for hypothermia during whole surgery and recovery process. One hour after injury, one dose of Dex (100 μg/kg) was IP administrated to rapidly induce early moderate to modest therapeutic hypothermia (30-32° C.). All mice body temperature were measured by rectal probe every 30 minutes to monitor the hypothermia after surgery till the mice fully waked up and back to normothermia. After SCI injury, at different time, multiple behavioral tests, were applied to examine functional outcomes and the potential neuroprotective effects of Dex treatment (FIG. 3A). The Basso Mouse Scale (BMS) was a reliable measurement that developed to examine the locomotion in spinal cord injured mice. It has been widely used in mouse functional evaluation and serves as an indicator of recovery. This test examines different locomotor categories such as paralysis, weight support, stepping, trunk stability, and so on. The scores ranged from 9 to 0 that represent normal movement to complete paralysis, respectively. Three days prior surgery, the BMS scores of all mice in different groups were measured. Only mice with 9 score were kept. The mice with score lower than 9 were replaced to make sure mice in different groups had same baseline of locomotion. After surgery, the BMS score was repeatedly measured at 3 days, 1 week, 2 weeks, 3 weeks, 4 weeks, 5 weeks, and 6 weeks (FIG. 3B). The result showed that sham surgery only slightly affected the mice locomotion with few of them scored 8 in BMS test on day 3 post-injury. On the contrary, SCI had a drastic impact on the locomotion in injured mice with most of them were in complete paralysis status and scored 0 in BMS test (FIG. 3B). Dex treated mice did not show the significant difference comparing to SCI only group at this time point. One week after surgery, all sham mice were back to the normal. While, both SCI and SCI+Dex group of mice showed same level of spontaneous recovery from SCI shock with average BMS score close to 5 (FIG. 3B). Thereafter, compared to the no significant change of BMS score in SCI only group, the BMS score of Dex treated SCI mice increased gradually from week 2 to week 6 and reached as high as about 7.8 (over 65% increase of BMS score compared to SCI only group) at the end of test. This result strongly suggested that Dex with its induced therapeutic hypothermia greatly improved the recovery of locomotion in SCI mice.

T9 contusive SCI will affect the injured mice hindlimb function. Thus, Rotarod was further performed to assess motor coordination, balance, and motor learning specifically post-injury. Only mice scored 5 and above in BMS can stay on the rod, therefore, the initiation of Rotarod test was 3 weeks after SCI according to BMS test result and repeated weekly till week 6. Three days pre-injury, the baseline of retention time in Rotarod test were measured with average around 40 sec among different group of mice. Sham mice learned and acquired the skill to stay on rod longer showing as 3 weeks after surgery with the retention time significantly increased from 41.7±0.78 sec to 59.8±5.2 sec (FIG. 2, c, n=10, p<0.01) and maintained the same level thereafter crossed all test time period. SCI had dramatically impact on Rotarod test with retention time dropped significantly from 39.1±1.3 sec to 9.1±2.4 sec (FIG. 3C, n=10, p<0.01) at 3 weeks post-injury. Then, the retention time of SCI mice in Rotarod test increased slightly, and gradually reached 23.1±3.4 sec at week 5 after injury (FIG. 3C). Interestingly, at week 6 post-injury, the retention time of SCI mice dropped again, which matched the time of second wave of neuroinflammation happened after SCI. Dex treated SCI mice showed same pattern as SCI only mice in Rotarod test, but had significant higher retention time compared to its cohort in each test time point. The increased rate was around 75% (n=10 for each group, p<0.05).

In addition to Rotarod test, Grid walk test further was ran twice at 4 and 6 weeks post-injury to assess hindlimb movements involved in sensorimotor functions, such as precise stepping, coordination and accurate placement of the paw. A one-foot drop error was counted when the hindlimb paw was entirely dropping through the grid with all toes and heel below the grid surface. The counting results showed that sham mice barely had foot fall, but SCI dramatically increase the fault rate to 55.3±21.1% at 4weeks post-trauma. At 6 weeks, the foot fall rate reduced limitedly in SCI mice (40.4±21.7%). Meanwhile, Dex treatment significantly decreased the foot fall rate at both test time point (SCI vs. Dex+SCI, 55.3±21.1% vs. 17.3±5.1% and 40.4±21.7% vs 12.9±3.6%, n=10, p<0.01). (FIG. 3D). Taken together, all these findings confirmed that Dex treatment greatly improved functional outcomes, particularly motor function after SCI.

### Example 4: Impact of Dexamedetomidine on Bladder Function After SCI

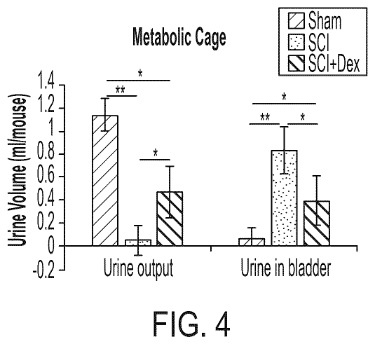

A metabolic cage experiment, which allows metabolic monitoring of mice in an automated and synchronized manner, showed that Dex significantly improved the bladder function following a SCI as compared to no hypothermia treatment, as shown in FIG. 4. The volume of urine of the animal was significantly reduced in 24 hours of injury. (sham group: 1.14±0.15 ml vs. SCI group: 0.06±0.13 ml, p<0.01, n=5). Dex treatment markedly enhanced the urination volume (Dex group: 0.48±0.22 ml vs. SCI group, p<0.05, n=5). SCI resulted in an increase of the residual urine volume (sham group: 0.08±0.08 ml vs. SCI group: 0.84±0.21 ml p<0.01, n=5) which was significantly reverted by Dex treatment (Dex group: 0.4±0.22 ml vs. SCI group p<0.05, n=5).

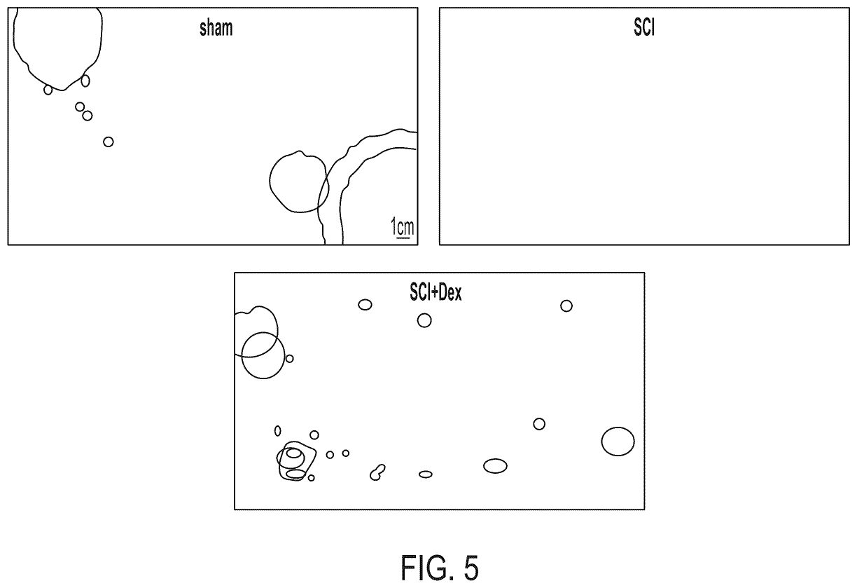

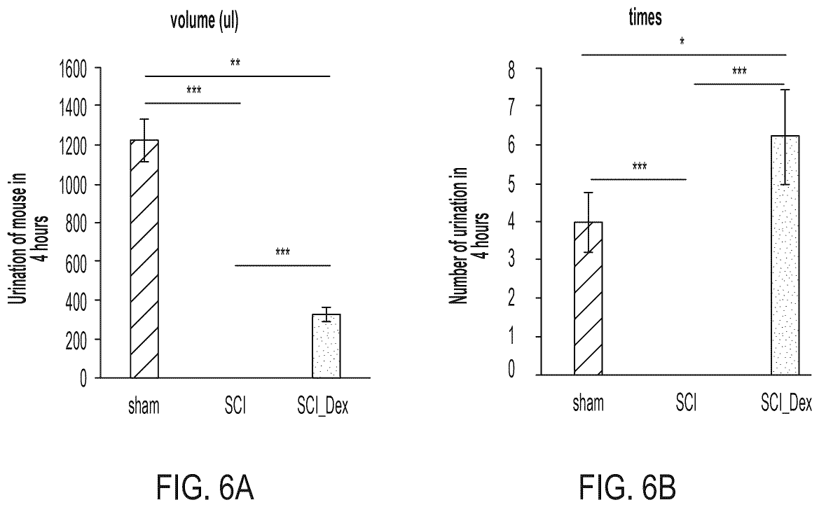

Results from a void spot assay (VSA), shown in FIG. 5, which involves placing a mouse in an enclosure lines by filter paper and quantifying the resulting urine spot pattern, showed that in 4 hours after 1 ml saline IP injection, sham mouse has 1.42±0.11 ml urine and an average frequency of urination of 4.5±0.58. SCI reduced the urination to 0 both in volume and frequency. Dex treatment significantly enhanced the urine volume (0.3±0.04 ml p<0.01 vs. SCI group, n=5) and frequency (6±0.82, p<0.01 vs. SCI group, n=5) (FIGS. 6A and 6B).

Referring to FIGS. 5A-5C, to evaluate the coordination of bladder and external urethral sphincter, urodynamic recordings were performed. Bladder cystometrograph (CMG) and external urethral sphincter electromyography (EMG) were co-recorded. The peak intravesical pressure in CMG indicates the contraction of bladder detrusor. The bursting signal in external urethral sphincter EMG is a sign of relaxation of external urethral sphincter. The coordination of bladder and external urethral sphincter was reflected by the synchronization of two signals. In sham group, two signals had a good synchronization with a 1:1 ratio. SCI significantly reduced the number of bursting signal and increased the non-voiding contraction of bladder (elevation of intravesical pressure without the relaxation of external urethral sphincter which failed to release the urine). Dex treatment enhanced the synchronization. In chronically injured mice, restoration of reflex external urethral sphincter relaxation correlates with the re-appearance of external urethral sphincter bursting. Therefore, external urethral sphincter bursting is a good indicator of external urethral sphincter functional recovery after SCI in mice. SCI reduced the voiding efficiency (sham 38.6±7.4% vs. SCI 3.8±2.4%, p<0.001, n=5).

Dex treatment significantly enhanced the voiding efficiency (Dex 15.9±6.6% vs. SCI group, p<0.05, n=5). Bladder capacity in the sham group was 0.22±0.05 ml. SCI dramatically increased the bladder capacity up to 0.98±0.14 ml (p<0.001 vs. sham). Dex treatment reduced the bladder capacity down to 0.56±0.31 ml (p<0.05 vs. SCI group). This improvement of coordination of bladder and external urethral sphincter might be attributed to the spared spinal cord tissue containing ascending and descending tracts particularly those spinobulbospinal reflex pathway responsible for micturition reflex. During the urodynamic recording, brain stem neurons controlling the urination within the spared supraspinal circuitry were activated and expressed c-fos. Significantly more c-fos positive neurons in raphe nuclei had been detected in Dex treatment groups than in SCI group) indicating more supraspinal circuities has been spared by Dex.

### Example 5: Impact of Dexamedetomidine on Bladder Function After SCI

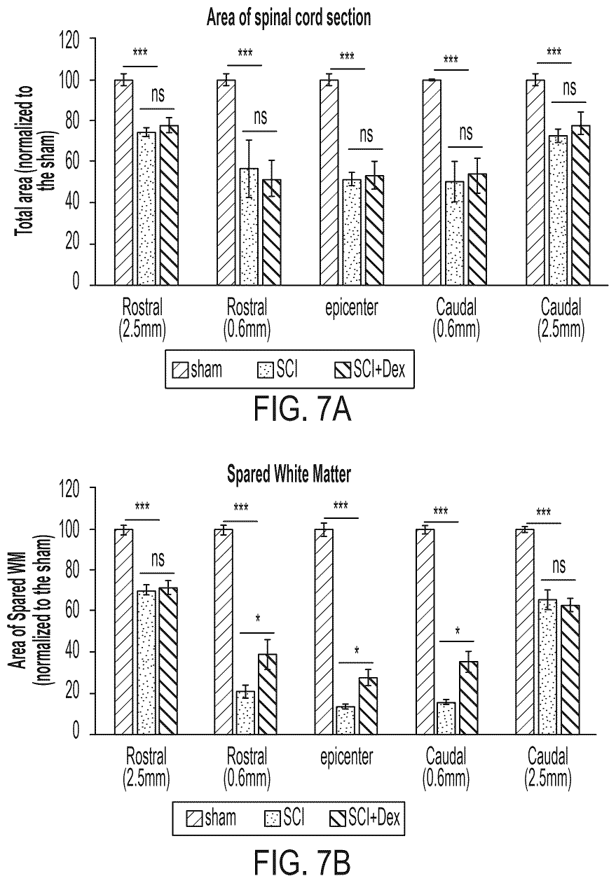

Behavior tests indicated that Dex might have great neuroprotective effects on SCI. We therefore further performed a battery of immunohistological assays to determine whether Dex treatment also improved pathological changes after SCI. We first measured the lesion size and spared white matter using cresyl violet and cosin staining. The results demonstrated that SCI not only destroyed most tissue in the epicenter and its surrounding area of injured mice, but also affected the remote area even 2.5 mm away from impact site. Compared to the sham control, the total area of spinal cord shrinked about 50% (FIG. 7A, spared total tissue area SCI vs. sham 51.7±1.9%, p<0.001, n=3) in the epicenter and its surrounding area (FIG. 7A, Rostral 0.6 mm 56.2±3.3%, and Caudal 0.6 mm 50.1±0.8%, p<0.001, n=3). Even in the remote region, the shrinkage of spinal cord was still significant (FIG. 7A, Rostral 2.5 mm 74.5±2.4%, and Caudal 2.5 mm 72.6±4.1%, p<0.001, n=3). In the impact site, the clear spared white matter area (pink staining) was barely detected. Most of them were mixed with blue staining. On the other hand, though the shrinkage pattern was similar in SCI+Dex group comparing to SCI only group, Dex treatment did preserve more white matter showing as a large clear pink staining area in the out layer of spinal cord both in epicenter and its surrounding area. Quantification data showed that over two times of white matter was preserved with Dex treatment (FIG. 7B, SCI vs. SCI+Dex, 13.4±3.1% vs 27.6.4±0.9%, P<0.05, n=3). More interestingly, butterfly structure of grey matter (blue staining) was also roughly preserved in Dex group, suggesting the survival of neurons even in epicenter of injured spinal cord with the treatment of Dex. In the per-epicenter region (0.6 mm), the clear butterfly structure of grey matter could be observed in spinal cord of Dex treated SCI mice. More spared WM and more survived neurons implied the better functional outcomes, which is strongly support our findings in behavior tests.

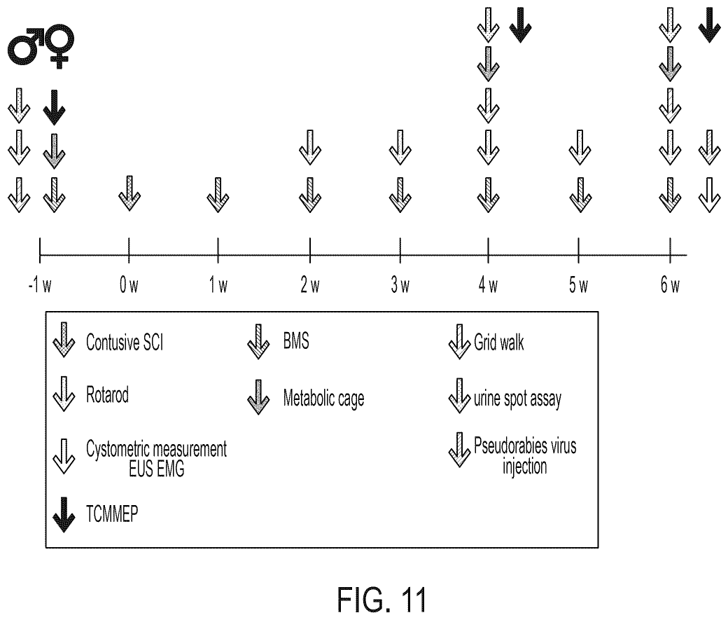

A summary timeline of that testing is shown in FIG. 11

### Example 6: Impact of Dexamedetomidine on Neurons and Axons After SCI

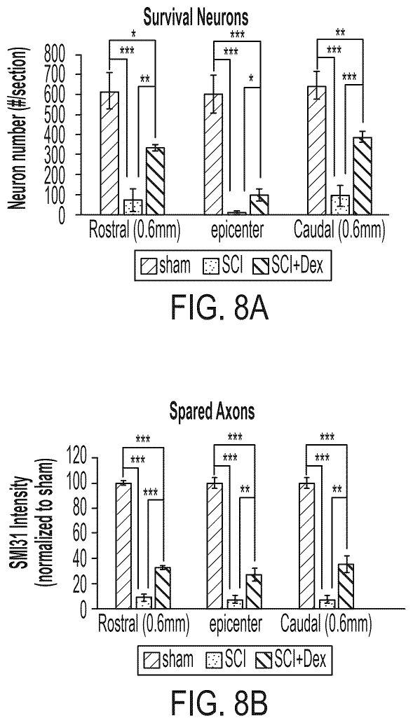

The preservation of butterfly structure in Dex treated animals suggested the survival of neurons in epicenter. To confirm this hypothesis, antibody against NeuN, a neuronal marker was used for immuostaining to detect in grey matter butterfly. SCI destroyed butterfly structure in epicenter and periregion. NeuN-positive signal was barely detected in this area, suggesting most of neurons got killed (FIG. 4, a and b, SCI). Dex treatment turned this over. Remarkable NeuN-positive neurons were detected even in epicenter and the number increased significantly in peri-region. We further counted the number of NeuN-positive neurons in each section. The quantification data confirmed that Dex treatment helped about 10 times of neurons survive after SCI compared to none-treatment group (FIG. 8A, SCI vs. SI+Dex). Dex treatment also preserved more white matter where axon track resided. Therefore, SMI31, an axon maker immunostaining was performed to evaluate the axons in spared white matter. In low power image, high intensity of SMI31 signal was detected in the white matter of sham mice (FIG. 8B, sham). SCI dramatically decrease both intensity and the area of SMI31 signal, while Dex treatment significantly preserved more of them (FIG. 8B, SCI vs. SCI+Dex). High power image of white box in low power clearly demonstrated the decrease of SMI31-positive puncta (cross section of axon) in injured mice had been reversed by Dex treatment (FIGS. 8A and 8D, SCI vs. SCI+Dex). SMI31-positive signal quantification result confirmed the above findings. Altogether, more neurons and axons spared with Dex treatment after SCI that might have great contribution to the improvement of functional outcomes.

### Example 7: Impact of Dexamedetomidine on Astroglia Scar Formation After SCI

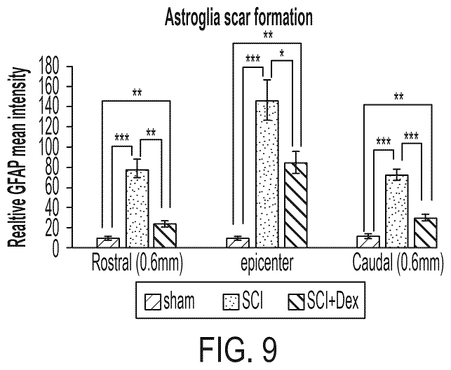

Reactive astrocyte is the one of major components of glia scar. The activation of astrocyte could be used as indicator for damage level. Thus, GFAP, a marker for astrocyte activation was used to monitor reactive astrocyte. Immunostaining with antibody against GFAP showed that, in sham mice, basal level of GFAP signal was detected mainly in outer layer of white matter. SCI induced strong astrocyte activation reflected by a dramatic increase of GFAP signal intensity to an extremely high level in epicenter. Over there, strong GFAP-positive reactive astrocyte formed a dense ring structure (scar formation) surrounding a GFAPnegative mass to confine the spread of damage. It has been reported that infiltration immune cells, fibroblast, and epidermal cell are major components of this GFAP-negative mass. In the peri-region of injury site, the structure of white matter and grey matter was destroyed, and two different matters were mixed together. Thus, strong GFAP-positive signal was detected evenly crossed whole spinal cord section. In the Dex-treated mice, the intensity of GFAP-positive signal decreased significantly in the epicenter compared to the none-treatment SCI mice. No dense ring structure was detected as well. In the peri-region, the GFAP distribution pattern of Dex treated mice was almost same as sham control with only a few small areas having comparable higher GFAP signals. GFAP intensity quantification further confirmed that Dex treatment significantly decreased GFAP signal compared to the none-treatment SCI group. (FIG. 9) These results suggested that Dex treatment might ameliorate damage and attenuate astrocyte activation and scar formation.

### Example 8: Comparison of Dexamedetomidine with Conventional Hypothermia Treatment After SCI

Systematic hypothermia has been suggested as a neuroprotective strategy to limit secondary injury in both animal models and human patient. However, using traditional methods of inducing systemic hypothermia, the meta-analysis and systematic review indicated that with hypothermia alone, the average improvement of locomotor function was limited (only about 24.5%) 16 and mostly transient (1-3 weeks after injury). Contrarily, Dex treatment in BMS test showed much stronger and longer improvement of locomotor function (70% vs. 25%). In order to evaluate whether the superior results seen in the Dex treatment group was due to the lab-dependent variations in injury model and severity, we performed a side-by-side accurate comparison to evaluate the neuroprotective efficacy between Dex treatment and conventional hypothermia (CH) treatment after SCI.

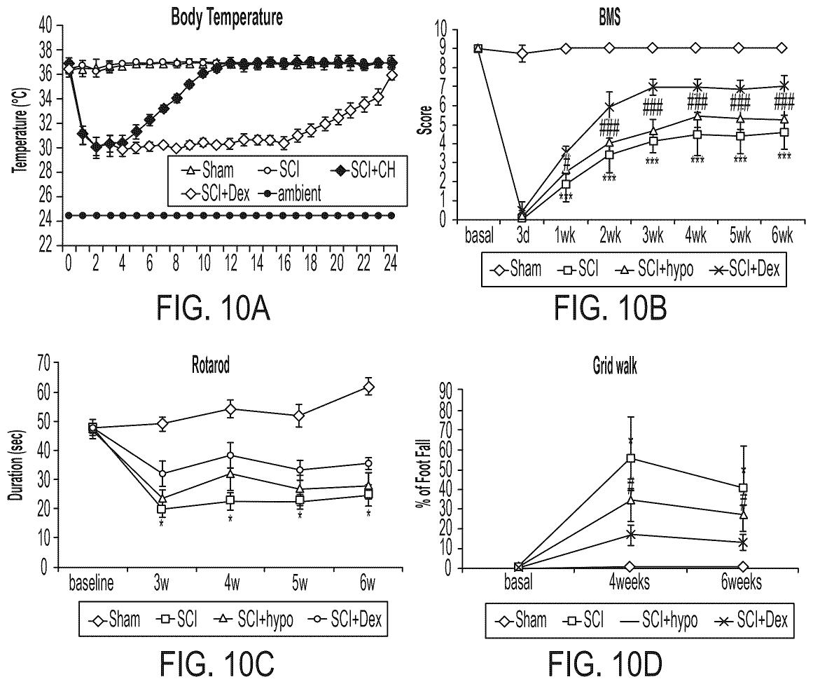

Conventional hypothermia achieved designed temperature using cooling-blanket or ice-pad. The general paradigms used for conventional hypothermia in animal (mostly rat) are applying 32-34° C. target temperature within one hour after injury, with 4 hours duration and 1° C. per hour re-warming rate. Infinite Horizon impact (IH), a more broadly and well accepted SCI injury model was used to generate contusive T9 moderate SCI. Young adult female mice (8-10 weeks old) with injury were randomly assigned into two groups which were treated at one hour post-trauma by either cooling blanket (SCI+CH) or Dex (100 μg/kg, SCI+Dex) at ambient normal room temperature of 24.5° C. to induce hypothermia. Sham and none-treatment SCI mice were put on the heating pad to maintain their normal body temperature. The body temperature was measured every 30 min using rectal probe to monitor hypothermia. Conventional hypothermia (moderate) was successfully achieved for 4 hours using cooling blanket (FIG. 10A). During this process, severe shiver was observed in all treated animals. As expected, Dex treatment induced 16 hours long moderate hypothermia in IH model (FIG. 10A). After injury, a same set of behavior tests used in LISA model were performed to determine whether Dex treatment had superior neuroprotective effects than CH. No surprising, the injury severity is similar between LISA and IH model, which is reflected by similar BMS score pattern (0-5) SCI only mice acquired via spontaneous recovery in both models (FIG. 10B). Dex treatment still had a good performance in IH model to gradually elevate BMS score from 0 over 7 at the end of BMS test (FIG. 10B, SCI vs. SCI+Dex, 4.6±0.89 vs. 7.13±0.48, n=10, ***p<0.005, increase rate 56%). CH showed the trend of neuroprotection, however, comparing to SCI group, the difference is not significant in 10 mice scope (FIG. 10B, SCI vs. SCI+CH, 4.6±0.89 vs. 5.3±0.57, n=10, ns, increase rate 15%). In BMS test, Dex treatment overwhelms the CH treatment (FIG. 10B, SCI+CH vs. SCI+Dex, 5.3±0.57 vs. 7.13±0.48, n=10, ###p<0.005). Same pattern was found in Rotarod test. Compared to none-treatment SCI mice, Dex treatment significantly improved motor function with average 55% increase rate. While, CH slightly increased the duration time with no significant difference (FIG. 10C, 6 wk, SCI Vs. SCI+CH vs. SCI+Dex, 24.6±3.5 vs. 27.7±4.64 vs. 35.3±1.84, n=10, SCI vs. SCI+Dex, *p<0.05). CH treatment had better performance in Grid walk test. Yet, no statistical significance reached comparing to SCI only group (FIG. 10D, 6 wk, SCI Vs. SCI+CH, 40.4±21.7 vs. 27.5±9.9, n=10). Meanwhile, Dex treatment dramatically reduced the foot fall compared to both SCI and SCI+CH group (FIG. 10D, 6 wk, SCI Vs. SCI+Dex, 40.4±21.7 vs. 12.9±3.6, n=10, *p<0.05, and SCI+CH Vs. SCI+Dex, 27.5±9.9 vs. 12.9±3.6, n=10, #p<0.05). The results from 3 different behavior tests all indicated that Dex treatment had superior neuroprotective effects than CH.

### Materials and Methods

Contusive spinal cord injury: Adult female C57BL/6 mice (8 weeks old, 18-22 g) were purchased from The Jackson Laboratory (Bar Harbor, ME). Depending on different injury model, mice were either randomly divided into three groups: sham, SCI+heating pad, and SCI+Dex for LISA injury model, or were randomly divided into four groups: sham, SCI+heating pad, SCI+Dex, and SCI+Conventional hypothermia for Infinite Horizons (IH) model. Sham-operated mice received the same surgical procedures, including laminectomy, as the SCI groups although they did not sustain an SCI. All animal care, handling, and surgery were performed in accordance with the Guide for the Care and Use of Laboratory Animals (National Research Council) and the Guidelines of the Institutional Animal Care and Use Committee of Indiana University School of Medicine. Spinal cord contusion was made at the thoracic (T9) vertebral level. After avertin anesthesia, animals will be placed on a thermometer-controlled heating pad for the duration of recovery (except Dex and conventional hypothermia treated group). For LISA injury model, C57/6J mice were anesthetized. A midline skin incision was made to expose the thoracolumbar spine, followed by the removal of the T9 spinous process and lamina to expose the dura mater. The displacement was set to be 0.5 mm. The T9 contusion injury was performed using a LISA impactor, where the injury level was determined using a laser sensor. After injury confirmation, the surgical area is sutured, the animal is hydrated and monitored for post-operative care, including analgesia and bladder expression. For IH model, a 1-inch skin incision was placed on the dorsal midline, musculature was retracted, and then the spinal column was visualized and placed into a stabilizer with two clamps retracting the spinous processes of the vertebrae directly surrounding the injury site. A laminectomy was performed to expose the dura. The spinal cord was contused using an Infinite Horizon Impactor (IH, Infinite Horizons, Lexington, KY) with a 0.5-mm diameter tip at an impact force of 60 kdyne. After injury, the U-shaped stabilizer was detached from the stage, and the mouse was removed from the stabilizer. The injury area was checked and, if present, bleeding was stopped. The muscles and skin were sutured in layers with 3-0 silk surgical suture (Henry Schein, Melville, NY). The injured mouse received subcutaneous (SC) injection of Meloxicam SR (5 mg/kg every 24 h for 72 h) for analgesia and 1 mL saline for hydration. The mouse will be placed in a temperature-and humidity-controlled cage and the body temperature was closely monitored until it recovered from anesthesia. Soft food and water was provided during recovery. Manual bladder expression was performed twice daily until reflex bladder emptying was established.

Behavioral assessments: Three behavior assessments including Basso Mouse Scale (BMS), grid-walking, Rotarod tests were employed. Three days prior to surgery, each mouse was trained in the open field 4 min for BMS, 3 trails for Rotarod, and 3 min on the grid for grid-walking. Baseline scores of the three assessments were recorded.

BMS locomotor test: The BMS test was performed weekly up to 6 weeks post-SCI to examine the animal general movement (locomotion) according to a method published previously. Briefly, each mouse was placed in an open field (diameter 42 inch) and observed for 4 min by two observers who were blinded to the experimental groups. The movements were scored based on the joint movement, weight support, plantar stepping, paw position, coordination, and trunk/tail control following the standard guide. The scores ranged from 9 to 0 that represent normal movement to complete paralysis, respectively. BMS was recorded before injury and at 3, 7, 14, 21, 28, 35, and 42 days post-injury.

Rotarod test: Rotorod is used to assay the hindlimb function of the animal. The animal was put on a rotated device. It consisted of a metal frame with a motorized, rotating assembly of rods. Animals were allowed to remain stationary. The rotational speed was slowly increased from 0 to 30 rpm within 90 sec. If animal skilled, it can remain on the device at this speed for extra 30 sec until the 2-min test period elapsed. Normally, a trial was ended if the animal fell completely off the rod or gripped the rod and spun around without attempting to walk on the rod. Mean duration on the rod was recorded.

Grid-walking: The grid-walking test assesses the ability of a mouse to place its paws on the rungs of a grid accurately during spontaneous exploration. An elevated metal rectangle grid (12×36 inch2 with each grid cell 0.5×0.5 inch2) and 16 inches above a table top was used for the hindlimb drop error test. Each mouse was placed in the center of the grid for 3 min. A one-foot drop error was counted when the hindlimb paw was entirely dropping through the grid with all toes and heel below the grid surface. The total number of foot drop errors and total number of steps were recorded by video. Two observers who were blinded to the experiment analyzed the video and counted the steps. The grid-walking test was performed on the day before surgery and on days 21 and 35 post-SCI. Only those mice with consistent plantar stepping (BMS≥5) were tested on the grid.

Tissue Processing: All the animals were deeply anesthetized with Avertin and then perfused transcardially with cold saline, followed by a fixative containing 4% paraformaldehyde (PFA) in PBS. The spinal cord was removed, post-fixed overnight in PFA, and cryoprotected for 48 hours in 30% sucrose. Serial 30 μm thick coronal sections were cut using a cryostat (Leica CM 1950), mounted on slides and stored at −20° C. The sections were then processed for histopathological analysis.

Cresyl violet and cosin staining: A set of slides were first removed from the freezer and allowed to air dry for a minimum of 60 minutes at room temperature. Subsequently, the sections were treated with two 5-minute rinses in 10 mM PBS, followed by a 1-minute rinse in ddH2O. Next, slides were immersed for 20 minutes in Nissl stain, followed by two 5-minute rinses in ddH2O. Subsequently, slides were sequentially immersed in 90% ethanol (3 minutes), 95% ethanol (3 minutes), two changes of 100% ethanol (3 minutes each), and finally, three changes of 100% xylene (3 minutes each). Lastly, slides were mounted using Permount.

Immunofluorescent Staining: Immunofluorescent staining was carried out according to our previous reports. In brief, sections on the slides were rinsed in PBS 3 times (5 minutes each) and incubated in blocking buffer (0.1% Triton X-100, 1% bovine serum albumin, and 5% normal serum in PBS) for 1 hour at room temperature. Then, primary antibodies were added to blocking buffer and incubated with sections overnight at 4° C. After 3 washes with PBS (5 minutes each), sections were incubated with the secondary antibody for 1 hour at room temperature and then treated with 4′,6-Diamidino-2-phenylindole (DAPI) for 2 minutes. Finally, sections were washed 3 times with PBS (5 minutes each), dried, and mounted using Fluoromount G (Southern Biotech, Birmingham, AL, USA). Primary antibodies and their final concentrations were as follows: NeuN antibody (1:500, chicken, Abcam, ab134014), SMI31 antibody (1:1000, mouse, Millipore, NE1022), and GFAP antibody (1:1000, mouse, Sigma, G3893). Secondary antibodies from Jackson ImmunoResearch Laboratories Inc. were all applied at the same dilution of 1:1000.

Imaging: The images were taken at a primary magnification of 10×, 40×, and 63× using an invert microscopy system (Zeiss, Axiovert 200 M) interfaced with a digital camera (Zeiss, Axio Cam MRc5) controlled by a computer.

Fluorescent intensity measurement and cell counting: The intensity of SMI31 or GFAP signal was measured using ImageJ software (version 1.47; NIH, Bethesda, MD). Briefly, images with SMI31 signals or GFAP signals were captured using a Zeiss inverted microscope (Zeiss Axio 200, Oberkochen, Germany). The contour of spinal cord was drawn using ImageJ software and the total SMI31 signal or mean intensity of GFAP signal within the contour was measured. The total SMI31 signals of different groups were normalized to the sham control and compared. The mean intensity of GFAP signals was compared among different groups. Every single NeuN positive Neurons on the section were counted. The total number of neurons per section then was compared among different groups.

I claim:

1. A method for inducing hypothermia in a subject post-trauma, comprising administering an effective amount of an adrenergic agonist to the subject, thereby inducing hypothermia in the subject, wherein the adrenergic agonist is administered to the subject after the subject has suffered an injury.

2. The method of claim 1, wherein inducing hypothermia comprises reducing the subject's core body temperature to a hypothermic temperature of about 30° C.-35° C., 30° C.-34° C., 30° C.-33° C., 30° C.-32° C., 31° C.-35° C., 31° C.-34° C., 31° C.-33° C., 32° C.-35° C., 32° C.-34° C., or 32° C.-33° C., wherein each range is inclusive.

3. The method of claim 2, wherein the hypothermic temperature is maintained for 0.5 to 6 hours, 0.5 to 12 hours, 0.5 to 24 hours, 6 to 12 hours, 6 to 24 hours, 6 to 8 hours, 2 to 6 hours, or 3 to 5 hours, wherein each range is inclusive, after administration of an adrenergic agonist.

4. The method of claim 3, wherein after maintaining the hypothermic temperature, the method further comprises applying heat to the subject until the subject's core body temperature reaches 36.5-38.5° C., inclusive, or allowing the subject's core body temperature to reach 36.5-38.5° C., inclusive, without intervention.

5. The method of claim 1, comprising inducing hypothermia in a subject while the subject is in an ambient temperature environment in the range of 18° C. to 40° C., 18° C. to 37° C., 18° C. to 35° C., 25° C. to 35° C., 22° C. to 30° C., 22° C. to 27° C., 23° C. to 27° C., 24° C. to 27° C., or 22° C. to 25° C., inclusive.

6. The method of claim 1, wherein the injury comprises traumatic brain injury, a stroke, a spinal cord injury, or a combination thereof.

7. The method of claim 1, comprising administering the adrenergic agonist to the subject about 1 to 24 hours, 1 to 16 hours, 1 to 12 hours, 0.5 to 1 hour, 0.5 to 2 hours, 0.5 to 3 hours, 1 to 8 hours, 2 to 6 hours, 3 to 5 hours, or 0 to 60 minutes after the injury, wherein each range is inclusive.

8. The method of claim 1, wherein the adrenergic agonist comprises p-aminoclonidine, lidamidine, tizanidine, moxonidine, xylazine, detomidine, medetomidine, dexmedetomidine, a pharmaceutical salt thereof, an analog thereof, or a combination thereof.

9. The method of claim 1, wherein the adrenergic agonist comprises an alpha2-adrenergic receptor agonist.

10. The method of claim 9, wherein the alpha2-adrenergic receptor agonist comprises dexmedetomidine hydrochloride.

11. The method of claim 1, comprising administering the adrenergic agonist to the subject by parenteral administration, oral administration, mucosal administration, subcutaneous administration, intravenous administration, intramuscular administration, intranasal administration, or a combination thereof.

12. The method of claim 11, comprising administering the adrenergic agonist to the subject by intravenous infusion.

13. The method of claim 1, comprising administering the adrenergic agonist to the subject in a single dose or more than one dose.

14. The method of claim 1, wherein the subject exhibits improved motor function, cognitive function, sensory function, autonomic function, sexual function, or a combination thereof, after induction of hypothermia, relative to motor function and/or cognitive function after the injury and prior to induction of hypothermia.

15. The method of claim 1, wherein the subject is a mammal.

16. The method of claim 15, wherein the mammal is a dog, a cat, a horse, or a human.

17. A method of treating a post-trauma injury in a subject, comprising administering at least one of a single dose at 8 mcg/kg and multiple doses at 0.2 to 2.5 mcg/kg per hour of an adrenergic agonist to the subject, thereby inducing hypothermia in the subject, wherein the adrenergic agonist is administered to the subject after the subject has suffered an injury.

18. The method of claim 17, wherein inducing hypothermia comprises reducing the subject's core body temperature to a hypothermic temperature of about 30° C.-35° C., 30° C.-34° C., 30° C.-33° C., 30° C.-32° C., 31° C.-35° C., 31° C.-34° C., 31° C.-33° C., 32° C.-35° C., 32° C.-34° C., or 32° C.-33° C., wherein each range is inclusive.

19. The method of claim 17 wherein the injury comprises traumatic brain injury, a stroke, a spinal cord injury, or a combination thereof.

20. The method of claim 17, wherein the alpha2-adrenergic receptor agonist comprises dexmedetomidine hydrochloride.
